(19) United States
(12) Patent Application Publication
(10) Pub. No.: US 2021/0290206 A1
(43) Pub. Date: Sep. 23, 2021

(54) Vaginal Microbiota Collection Device Modular Preservation Unit

(71) Applicant: Christopher Stevens, Houston, TX (US)

(72) Inventor: Christopher Stevens, Houston, TX (US)

(21) Appl. No.: 16/319,513

(22) PCT Filed: Jul. 19, 2017
(86) PCT No.: PCT/US17/42765

§ 371 Date: Jan. 22, 2019

Related U.S. Application Data
(60) Provisional application No. 62/363,893, filed on Jul. 19, 2016.

(51) Int. Cl. A61B 10/00

(52) U.S. Cl. CPC A61B 10/0045; A61B 2010/0216; A61B 2010/0074

(57) ABSTRACT

The present invention discloses a vaginal microbiota collection device and modular preservation unit comprising a means to collect and preserve the vaginal microbiota from an individual, a means to distribute the harvested microbiota, or both. The means to distribute the harvested microbiota comprises a collection apparatus with one or more modular components. The vaginal microbiota is distributed based on need or validation. The collection and preservation of the vaginal microbiota is accomplished by a piston mechanism. The present invention also discloses a method of harvesting vaginal microbiota using the device disclosed herein.

### BACKGROUND OF THE INVENTION

### Field of the Invention

The present invention generally relates to a device and a method for use that would aid healthcare professionals in collecting and preserving vaginal microbiota. More specifically, the present invention is drawn to a device, namely, a vaginal microbiota collection device modular preservation unit, as well as a method detailing the use of this device, that will enable healthcare professionals to collect and preserve vaginal microbiota for investigational or therapeutic purposes.

### Background

The microbiome (a collection of micro-organisms that live on or within an environment) is increasingly becoming an area of study and interest within and among all levels of the medical community. Specifically, the vaginal microbiome is of particular interest to modern medicine as it harbors a patient-specific, temporally variable environment that plays an important role in states of health and sickness for not only women but also to their progeny. As has been postulated and advanced, the microbial makeup of the vaginal environment can have a profound effect on the development, physiology, immunity, and nutrition of individual woman (See generally Bing Ma, et al., 2012, The Vaginal Microbiome: Rethinking Health and Diseases, Annu Rev Microbiol. 2012; 66: pp. 371-389). Yet, of equal importance, the vaginal microbiome (via the birth canal) is the first source of bacterial inoculation of infants during the birthing process (i.e. vaginal seeding). This initial inoculation is bypassed when an infant is delivered via cesarean section (C-section) and these infants have an initial individual microbiome that more closely reflects skin and environmental microbiota than vaginal (See Phillips, 2009, Gut Reaction: Environmental Effects on the Human Microbiota, Environ Health Perspect. 2009 May; 117(5): A198-A205). Additionally, there is an increased incidence of chronic illness including but not limited to celiac disease, asthma, autoimmune disorders, obesity, and neurodevelopmental disorders due to changes in these infant's microbiota (See, also, generally Noel Mueller et al., 2014, The infant microbiome development: mom matters, Trends Mol Med. 2015 February; 21(2): pp. 109-117 and Moya-Perez et al., 2017, Intervention strategies for cesarean section-induced alterations in the microbiota-gut-brain axis, Nutr Rev. 2017 April; 75(4): pp. 225-240). Studies have demonstrated that the mother's vaginal microbiota can be transferred to infants born by C-section and the microbiome of these infants resemble more closely to that of infants born vaginally after such transfer (See Dominguez-Bello et al., 2016, Partial restoration of the microbiota of cesarean-born infants via vaginal microbial transfer, Nat Med. 2016 Mar. 22(3): 250-253). Thus, there is a long-felt but significant and un-met need in the art for a medical device and method for use that aids healthcare professionals in harvesting and/or preserving the vaginal microbiota, for investigational or therapeutic purposes, that is novel, practical and useful. The present invention satisfies this long-standing need in the art.

### DESCRIPTION OF PREFERRED EMBODIMENTS

The present invention provides a device (and method for use) that is intended to be an easy to use, and easy to access and manipulate, device that facilitates clean harvesting of vaginal microbiota. The advantages of this device include, but are not limited to, standardization of the harvesting process, preservation of the aliquot for testing/analysis, increased preservation of anaerobic bacteria (via a reduction in oxygen exposure), reduced environmental exposure/cross contamination, and the ability to effortlessly transfer the collected bacteria into a modular collection/preservation kit.

In a preferred embodiment, the present invention provides a device comprising a means to harvest and preserve vaginal microbiota and/or a means to distribute the collected vaginal microbiota into various modular attachments. As shown in FIG. 1, the means to harvest and preserve the vaginal microbiota comprises parts including, but not limited to, a break away seal (1) that keeps the circular flaps (2) in a closed compact position until activated; circular flaps (2) that open out (once activated) to direct fluid/flush into a collection reservoir (15). The circular flaps (2) may be made of materials that are flexible including, but not limited to, latex or rubber and form a relative seal with the walls of the vaginal canal (not depicted); a stopper cap (3) comprising a rubber based end cap that forms a plug at the end of the syringe piston and contains fluid ports to allow fluid rinsing/collection; a diluent out port (4) that distributes fluid injected into a fluid in/out port (13) into the vaginal canal; an injection and extraction in/out port (5) that allows collection of harvested microbiota from the collection reservoir (15) into a syringe (not depicted) attached to the fluid in/out port (13); a series of tapered sponge/gauze/brushes (6) that are tapered to prevent the stopper cap (3) from catching on a sponge/gauze/brush and blocking the ability to close completely; a hard walled, cylindrical chamber for the encapsulation of the operative mechanisms of the device (most closely resembling the barrel of a hypodermic syringe) (7); depth stopping wings (8) made of soft but firm rubber material and functions to prevent the device from becoming lodged or advanced too far into the vaginal canal; a plunger base (9) that remains in a fixed place at the base of the syringe chamber and allows the piston to extend and retract (similar to hydraulic piston with wiper seals that allow piston movement while keeping the contents internalized); a finger ledge (10) to facilitate grip and utilization of the device; a piston rod shaft (11) which is made to harbor a fluid catheter, a catheter lumen (12) that could be a single or a double lumen; a fluid in/out port (13) that connects to a standard Luer taper (e.g. “Luer Iok”®) type syringe and may have a dedicated in and out port; a piston rod base handle (14) that may be in the form of a ring and functions as a grip for piston rod shaft (11) and allows for the rotation of the collection apparatus while extended into the vaginal canal; and a collection reservoir (15) comprising an inner chamber that initially holds the collection apparatus and later holds the collected fluid/microbiota.

In another preferred embodiment, the means to distribute the harvested microbiota comprises a collection apparatus, where the collection apparatus has one or more modules, that is removably attached to fluid in/out port (13) or ports (not depicted). The collection apparatus of the device is as shown in FIG. 2. The collection apparatus includes, but is not limited to, a test or analyze port or vacutainer or sample bag (21), a wipe or gauze pack (22), a bulb type dispenser (23), a douche bag (24), a preservation bag (25), a centrifuge or cone (26), and nasal swabs (27).

In another embodiment, the module of the collection apparatus comprises a test or analysis port that allows for the extraction of an aliquot of vaginal fluid for preserved analysis via in/out port(s). The aliquot is extracted by means including, but not limited to, using a vacutainer that pulls fluid into a vacuum sample tube, collecting the aliquot in a volumetric sample container (with or without preservative), or both pulling fluid into the collection sample tube and collecting the aliquot into a volumetric sample container.

In still yet another embodiment, the module of the collection apparatus comprises a wipe/gauze pack that allows for an aliquot to be distributed into a sealed pouch containing sterile gauze. The gauze is then used to transfer the collected vaginal microbiota to the skin of an infant delivered via C-section, or other target area, thereby achieving the allocation of vaginal microbiota that the infant would have received through a vaginal delivery.

In another embodiment, the module of the collection apparatus comprises a bulb type dispenser that allows for an aliquot of vaginal microbiota to be collected into a bulb to facilitate droplet expression into a baby's mouth, into a bottle of milk, or as indicated by the healthcare provider's needs.

In yet another embodiment, the module of the collection apparatus comprises a douche bag that allows for an aliquot to be collected into a bag intended for the short or mid-term preservation with the intent of redistributing contents into the vagina via douche—the douche bag encompassing a preservative or an additive or no preservative or additive. The douche bag may be used to re-inoculate vaginal microbiota after a cycle of antibiotics, or a known period of vaginal dysbiosis or any other clinical indications.

In still yet another embodiment, the module of the collection apparatus comprises a preservation bag that allows for an aliquot of vaginal microbiota to be collected into a bag for long term preservation where the preservation bag may have the preservative or additive already present in the bag.

In further yet another embodiment, the module of the collection apparatus comprises a centrifuge/cone that allows an aliquot to be collected into a cone for centrifugation and liquid separation for concentration of the vaginal microbiota collected.

In yet another embodiment, the module of the collection apparatus comprises nasal swabs that allow for an aliquot to be collected into a sealed pouch containing cotton tipped applicators. The nasal swabs would facilitate the transfer of collected microbiota bacteria into the nasal cavity of the baby, or other target area, born via C-section.

As shown in FIG. 3, the device is activated by advancing the piston rod shaft (11) forward. The break-away seal (1) releases the circular flaps (2) out. The diluent is injected in through a port (13) or ports (not depicted) and the diluent can be expressed through the port (4) or ports (not depicted). The diluent and the microbiota are absorbed into a sponge or gauze (6). As shown in FIG. 4, when the piston rod (11) is retracted in the device, compression pulls the sponge or gauze (6) into the chamber (7). Additional diluent may be injected in the injection port(s) (13) and is expressed out of the port (s) (4) into the chamber reservoir (15). The device is then agitated to disperse or homogenize the collected vaginal bacteria. As shown in FIG. 5, the homogenized microbiota is retracted via the extraction in port (5) or ports (not depicted) via the fluid in port (13) or a plurality of ports (not depicted). A stopper (3) residing at the terminal end of the piston rod is pulled into the chamber (7) compressing the sponge or gauze (6) which (A) concentrates the collected vaginal bacteria and fluid and (B) forces the collected fluid to retreat through the extraction in port (5) or port(s) (not depicted). The solution is then collected into a syringe attached to the port (13) or port(s) (not depicted).

In another preferred embodiment, there is a method of use, and techniques for using the same, of the proffered device to collect the vaginal microbiota. Such a method comprises inserting the device to the depth stop, advancing the piston forward such that the break-away seal releases the circular flaps in an outward direction, injecting diluent through one port, or a plurality of ports, expressing out the diluent through the same or another port, or plurality of ports, allowing the diluent and microbiota to be absorbed into the sponge or gauze located within the device, pulling the sponge/gauze into the chamber by retracting the piston rod, injecting the diluent in through one port, or a plurality of ports, and expressing the diluent out into the chamber reservoir, agitating the device to homogenize or disperse the microbiota, retracting the microbiota out via a port, or ports, located in the device, pulling the stopper into the chamber and compressing the sponge/gauze and collecting the solution in the collection apparatus.

In yet another preferred embodiment, there is a “kit” comprising the device described above wherein all of the components are within one unit for individualized and mass distribution.

### DESCRIPTION OF DRAWINGS

FIG. 1 shows the means to harvest and preserve the vaginal microbiota of the device.

FIG. 2 shows a collection apparatus comprising of one or more modules.

FIG. 3 shows the device as it is inserted to depth stop.

FIG. 4 shows the device when the piston is retracted which pulls the sponge or gauze into the chamber.

FIG. 5 shows the device as the homogenized microbiota is retracted out via the extraction in port (5) or the fluid in port (13).

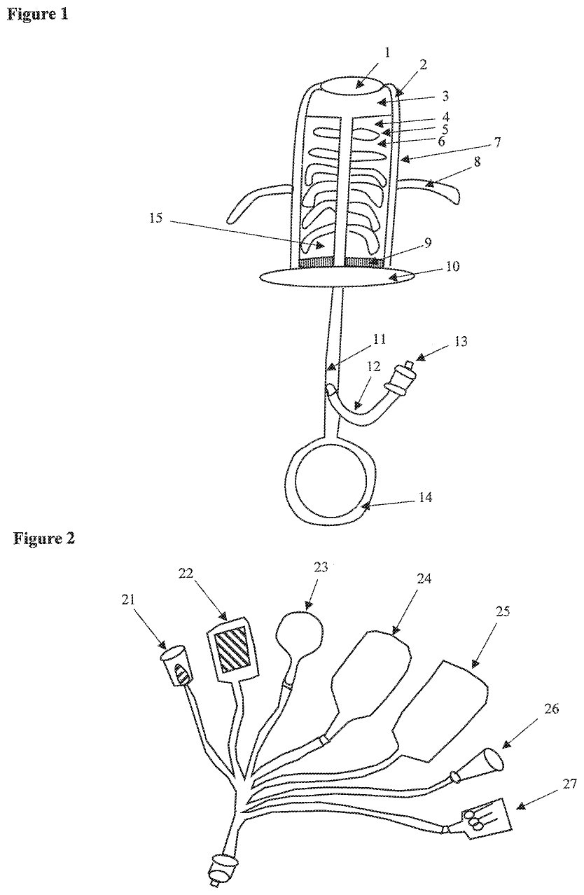

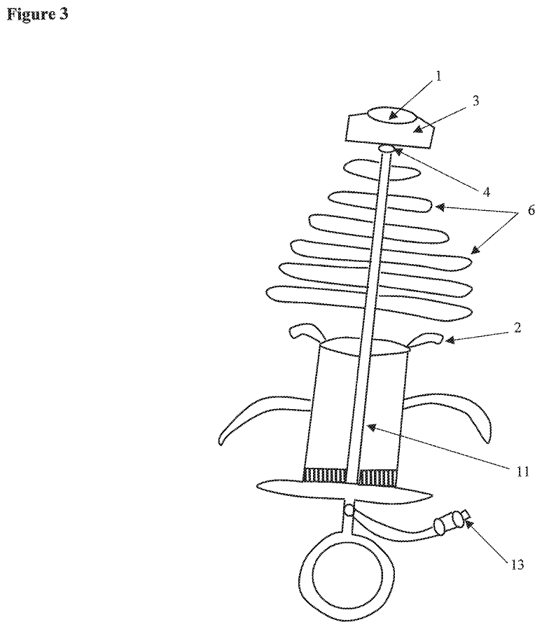

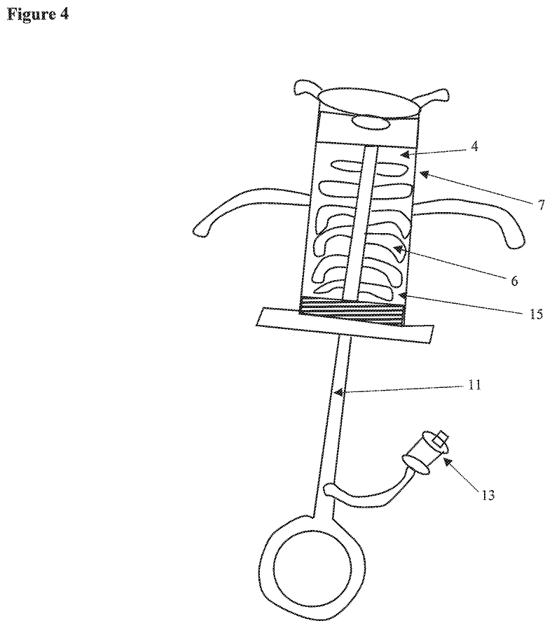

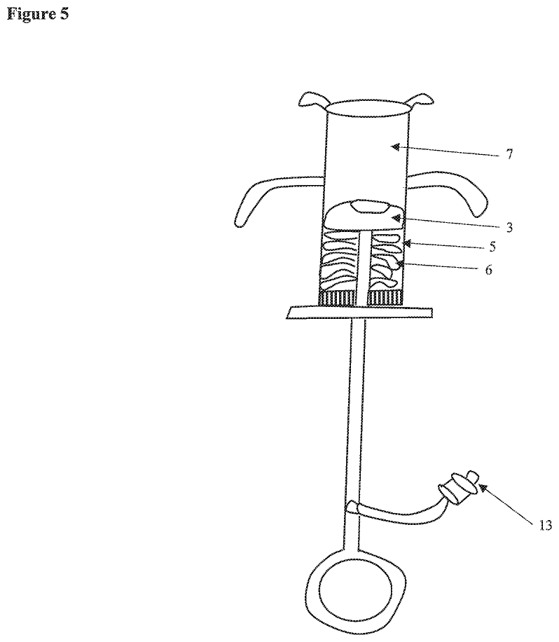

The foregoing descriptions of the embodiments of the present invention have been presented for purposes of illustration and description. They are not intended to be exhaustive or to limit the present invention to the precise forms disclosed. The exemplary embodiments were chosen and described in order to best explain the principles of the present invention and its practical application, to thereby enable others skilled in the art to best utilize the present invention.

I claim as my invention:

1. A device for collecting and preserving vaginal microbiota comprising:
a hard-walled cylindrical chamber, resembling a syringe barrel or a piston cylinder, which is closed at the most proximal portion and open at the most distal portion for the encapsulation of the operative mechanisms of the device;
depth stop wings affixed to the exterior portion of said hard-walled chamber that are positioned roughly equidistance from the proximal and distal portions of said hard walled chamber at a depth that allows the device to reach a predetermined depth within the vagina without becoming lodged or advanced too far within the vaginal cavity;
a break away seal crowning the most distal portion of the device serving the dual function of (a) sealing the device and (b) maintaining the circular flaps in a closed position prior to device activation and utilization;
circular flaps, made of a flexible material (e.g. latex or rubber), which are attached to the outer circumference of the most distal portion of the hard-walled cylindrical chamber and form a relative seal with the walls of the vaginal canal;
a disc-shaped finger ledge residing at the most proximal portion of the hard-walled capsule which forms the closed portion of said capsule, contains at its center an orifice capable of accepting a piston rod, and which acts to facilitate grip and opposing actions of release (insertion) and withdrawal of the inner mechanisms of the device;
a plunger base that remains in a fixed place at the base of the syringe chamber and allows the piston rod to extend and retract (similar to hydraulic piston with wiper seals that allow piston movement while keeping the contents internalized);
an operative mechanism in the form of a piston rod within a hard-walled cylindrical chamber that moves within the hard-walled cylindrical capsule to facilitate vaginal bacteria collection through reversible release into the vaginal cavity and subsequent retraction back into the hard-walled cylindrical chamber;
said operative mechanism piston rod comprising, from proximal to distal portions, a piston rod base handle, a piston shaft (lying partially outside and partially inside of the hard-walled cylindrical capsule), an internalized lumen cavity connecting an externally residing in/out port, or a plurality of ports, with an internally placed insertion/extraction portal, or a plurality of portals, a plurality of tapered appendages, and a stopper cap at the terminal end of the piston rod which acts within the hard-walled cylindrical capsule, in concert with the piston rod, as a plunger, to aid in the advancement of the piton rod into the vaginal cavity, limit fluid egress upon collection and create the necessary force and pressure to facilitate vaginal fluid harvesting;

2. The piston rod as described in claim 1, further comprising:
said piston rod containing within it an encapsulated, internalized lumen or cavity that runs the length of the piston rod and may accept a single, double, or multiple lumen fluid catheter that runs the length of the piston rod and is reversibly accessible from a fluid in/out port, or a plurality of ports, attached to a point lying externally between said finger ledge and the piston rod base handle;
said fluid in/out port, or plurality of ports, that is/are equally functionally accepting of a syringe (e.g. “Luer Lok”® type syringe), modular collection device, or similar means of fluid extraction and collection;
said piston rod where the rod is made to traverse an orifice existing at the base of the hard-walled capsule which is encircled by said finger ledge and an adjacent plunger base which remains fixed at the base of the hard-walled capsule, “syringe” base and allows the unsinuous movement (i.e. extension and retraction) of the piston rod;
said piston rod that, along its internalized portion, has attached to its shaft a plurality of tapered, fluid absorbing appendages, in the form of sponges, gauze or brushed, that have the inherent property of absorption to facilitate the collection of vaginal fluid from the vaginal canal;
said piston rod that terminates in a stopper cap containing a single fluid port or a plurality of fluid ports to allow fluid rinsing and fluid collection that further serves to block the retreat of collected fluid and reversible creates compression upon said tapered syringes to facilitate collection of vaginal fluid via said piston rod internalized lumen and said in/out port(s);

3. The device of claim 1, wherein said stopper cap, comprises an end cap that forms a plug at the end of the syringe piston rod and contains a fluid port, or a plurality of ports, to allow for fluid injection rinsing and vaginal fluid collection and is made of rubber or other suitable material having the proper ratio of rigidity to flexibility.

4. The device of claim 1, wherein syringe piston rod contains an extraction port, or a plurality of extraction ports, that allows for the collection of microbiota from a collection reservoir residing at the base of the hard-walled cylindrical capsule which is comprised of an inner chamber that initially holds the collection apparatus and later acts as a collection receptacle for collected fluid/microbiota.

5. The device of claim 1, wherein the plurality of tapered, fluid absorbing appendages may be in the form of a sponge, gauze, or brush that are tapered to prevent the stopper cap, hard-walled capsule, or a combination of the two from blocking the complete closure of the device and thus obstructing the required compression and ultimate retrieval of vaginal fluids.

6. The device of claim 1, wherein the piston rod base handle is a ring, a disc running perpendicular to the piston rod shaft, or a similar attachment useful for insertion and retraction of the piston rod within the cylindrical barrel of the device by the device operator.

7. The device of claim 1, wherein the depth stop wings may be made of a soft but firm material that may be in the form of a circle, or semi-circle, or a set of unattached “wings” which may either partially or fully encircle the circumference of the hard-walled capsule at a point and distance from the most terminal end of the device that is commensurate with varying vaginal depths.

8. The device of claim 1, wherein the depth stop wings may be secured, reversibly secured, or movably adjustable based on vaginal cavity width and depth.

9. A modular collection apparatus wherein said fluid in/out port or ports may have the capability of reversible coupling to a “Lure Lok”® system or harvested microbiota collection apparatus comprising one or more of the following:
a test or analysis port;
a vacutainer or any sterile glass tube for fluid collection with a rubber stopper creating a vacuum seal inside of the tube facilitating the draw of a predetermined volume of liquid;
a sample bag;
a wipe/gauze pack;
a bulb-type dispenser;
a douche bag;
a preservation bag;
a centrifuge or cone; and/or
nasal swabs.

10. The device of claim 9, wherein the module of the collection apparatus comprises a test or analysis port that allows for the extraction of an aliquot of vaginal fluid for preserved analysis where the aliquot is extracted by means including, but not limited to, using a vacutainer that pulls fluid into a vacuum sample tube, collecting the aliquot in a volumetric sample container (with or without preservative), or both pulling fluid into the collection sample tube and collecting the aliquot into a volumetric sample container.

11. The device of claim 9, wherein the module of the collection apparatus comprises a wipe/gauze pack that allows for an aliquot to be distributed into a sealed pouch containing sterile gauze where the gauze is then used to transfer the collected vaginal microbiota to the skin of an infant delivered via C-section, or other target, thereby achieving the allocation of vaginal microbiota that the infant would have received through a vaginal delivery.

12. The device of claim 9, wherein the module of the collection apparatus comprises a bulb type dispenser that allows for an aliquot of vaginal microbiota to be collected into a bulb to facilitate droplet expression into a baby's mouth, into a bottle of milk, or other target area as dictated by the healthcare provider's needs.

13. The device of claim 9, wherein the module of the collection apparatus comprises a douche bag that allows for an aliquot to be collected into a bag intended for the short or mid-term preservation with the intent of redistributing contents into the vagina via douche where the douche bag may have a preservative or an additive already present in the bag and said douche bag may be used to re-inoculate vaginal microbiota after a cycle of antibiotics or a known period of vaginal dysbiosis or any other clinical validations.

14. The device of claim 9, wherein the module of the collection apparatus comprises a preservation bag that allows for an aliquot of vaginal microbiota to be collected into a bag for long term preservation wherein said bag may have a preservative or additive already present in the bag.

15. The device of claim 9, wherein the module of the collection apparatus comprises a centrifuge/cone that allows an aliquot to be collected into a cone for centrifugation and liquid separation for concentration of the vaginal microbiota collected where said collection apparatus may also contain an additive or preservative.

16. The device of claim 9, wherein the module of the collection apparatus comprises nasal swabs that allow for an aliquot to be collected into a sealed pouch containing cotton tipped applicators where the nasal swabs would facilitate the transfer of collected microbiota bacteria into the nasal cavity, or other target area of the baby, born via C-section.

17. The modular device of claim 9, wherein the device is available as a kit.

18. A method of vaginal microbiota collecting consisting of the following steps where:
A collection device is activated by advancing the piston rod shaft forward;
the break-away seal releases the circular flaps outward;
a diluent is injected in and expressed through a port or a plurality of ports;
said diluent and the vaginal microbiota are absorbed into the sponge or gauze;
the piston is retracted into the device;
the sponge or gauze is pulled into the chamber;
said diluent is injected in the injection port and expressed out of the port into the chamber reservoir;
the device is then agitated to disperse or homogenize the collected vaginal bacteria;
the homogenized microbiota is retracted via the extraction in port together with the fluid in port;
a stopper that is attached to the terminal portion of the piston rod shaft is pulled into the chamber; the sponge or gauze is compressed thereby concentrating the collected vaginal bacteria and fluid;
pressure created by the retraction of the device forces the collected fluid to retreat through the extraction in port; and
the solution is then collected into a syringe attached or a modular device to said port or a plurality of ports.

19. A method for use of the proffered device to collect the vaginal microbiota comprising:
inserting the device to the depth stop;
advancing the piston forward such that the break-away seal releases the circular flaps in an outward direction;
injecting diluent through one port, or a plurality of ports;
expressing out the diluent through the same or another port or ports;
allowing the diluent and microbiota to be absorbed into the sponge or gauze located within the device;
pulling the sponge/gauze into the chamber by retracting the piston rod;
injecting the diluent in through one port or a plurality of ports;
expressing the diluent out into the chamber reservoir;
agitating the device to homogenize or disperse the microbiota;
retracting the microbiota out via a port, or ports, located in the device;
pulling the stopper into the chamber;
compressing the sponge/gauze;
and collecting the solution in the collection apparatus via said port or ports.

20. The method of claim 19, wherein the depth stop is adjustable and is adjusted prior to insertion.
